(19) United States
(12) Patent Application Publication
(10) Pub. No.: US 2025/0270184 A1
(43) Pub. Date: Aug. 28, 2025

(54) PROCESSES FOR THE PREPARATION OF FINERENONE

(71) Applicant: TEVA PHARMACEUTICALS INTERNATIONAL GMBH, Jona (CH)

(72) Inventors: Vadivelan Rengasamy, Ariyalur District (IN); Sundaraselvan Ariyamuthu, Tirunelveli (IN); Mustapha Mandewale, Kolhapur (IN); Elluru Subbireddy, Greater Noida (IN); Gaurav Kapoor, Yamuna Nagar (IN); Soumya Mukherjee, Durgapur (IN); Dnyaneshwar Nighot, Pune (IN); Sandeep Kumar Kushwaha, Banda (IN)

(21) Appl. No.: 18/857,222

(22) PCT Filed: Apr. 18, 2023
(86) PCT No.: PCT/US2023/018968

§ 371 Date: Oct. 16, 2024

(30) Foreign Application Priority Data
Apr. 18, 2022 (IN) 202211022829

(51) Int. Cl. C07D 401/04

(52) U.S. Cl. CPC C07D 401/04

(57) ABSTRACT

The present disclosure encompasses processes for the preparation of (S)-Finerenone.

### FIELD OF THE DISCLOSURE

The present disclosure encompasses processes for the preparation of Finerenone and (S)-Finerenone.

### BACKGROUND OF THE DISCLOSURE

Finerenone, (4S) 4-(4-cyano-2-methoxyphenyl)-5-ethoxy-2,8-dimethyl-1,4-dihydro-1,6-naphthyridine-3-carboxamide, has the following chemical structure:

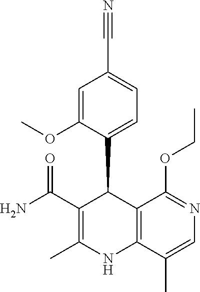 [SMILES: CCOc1ncc(C)c2c1[C@H](c1ccc(C#N)cc1OC)C(C(N)=O)=C(C)N2]

Finerenone is a nonsteroidal mineralocorticoid receptor antagonist, and it is indicated to reduce the risk of sustained eGFR decline, end-stage kidney disease, cardiovascular death, nonfatal myocardial infarction, and hospitalization for heart failure in adult patients with chronic kidney disease (CKD) associated with type 2 diabetes (T2D). In the U.S. it is marketed under the name KERENDIA® by Bayer Healthcare Pharmaceuticals Inc.

The compound is described in Int'l Publication No. WO 2008/104306.

Several processes for the preparation of Finerenone are known. For example, U.S. Pat. No. 8,436,180 discloses a process for Finerenone. Chiral resolution of racemic Finerenone is done by chiral chromatography.

Also, U.S. Pat. No. 10,059,707 and its continuation applications, U.S. Pat. Nos. 10,399,977 and 10,336,749, describe processes for Finerenone involving chiral chromatography for the preparation of the (S)-enantiomer.

U.S. Pat. No. 10,392,384 describes the preparation of racemic Finerenone from (R)-enantiomer of Finerenone. The described processes involve electrochemical reduction.

U.S. Patent Application Publication No. 2021/0163474 also describes the preparation of Finerenone. In this process chiral resolution of Finerenone is done by using chiral substituted tartaric acid esters.

Int'l Publication Nos. WO 2021074072 and WO 2021074078 describe processes for Finerenone involving chiral resolution of different cyanoester intermediates with aromatic tartaric acid derivatives.

Int'l Publication No. WO 2021/074077 describes an enzymatic process for the preparation of (S)-Finerenone.

CN 115340450 describes a process for Finerenone involving chiral resolution of a carboxylic acid intermediate with aromatic tartaric acid derivative.

CN 115340539 describes a process for Finerenone involving chiral resolution of an ester intermediate.

Thus, there is a need in the art for additional processes for the preparation of Finerenone, especially (S)-Finerenone that provide Finerenone in high quality and high yield, and that can be adapted to large (industrial) scale production.

### SUMMARY OF THE DISCLOSURE

The present invention provides processes for preparing Finerenone and more specifically (S)-Finerenone, which enable high yields and purity.

The present disclosure also provides intermediates, which can be used for the preparation of Finerenone or (S)-Finerenone.

### DETAILED DESCRIPTION OF THE INVENTION

In a first aspect, the present invention provides a process for the preparation of Finerenone according to Scheme 1.

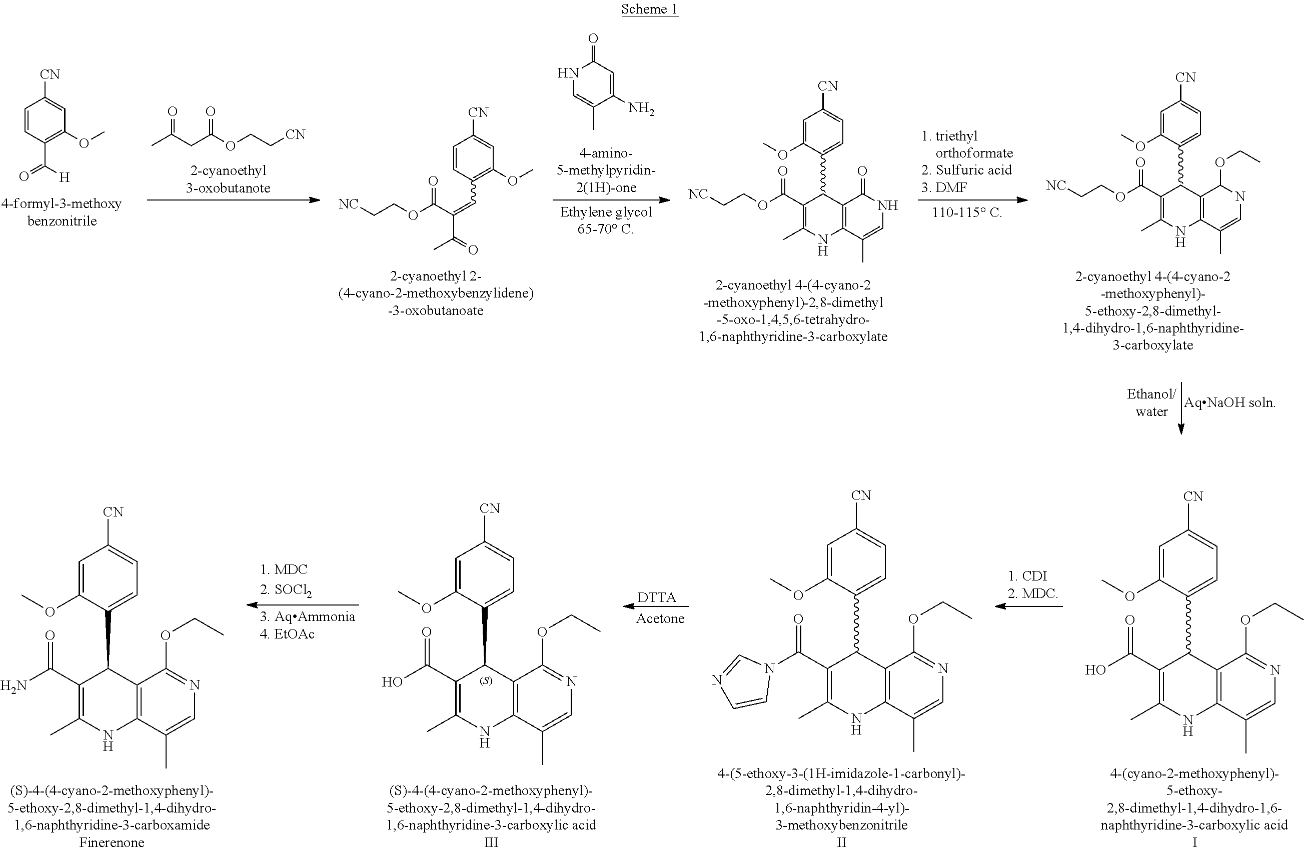

The process according to Scheme 1 involves the chiral resolution of a compound of formula II

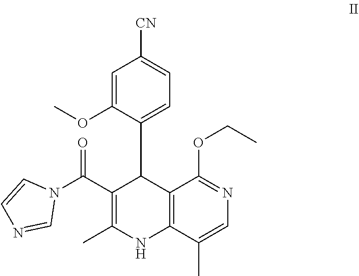 [SMILES: CCOc1ncc(C)c2c1C(c1ccc(C#N)cc1OC)C(C(=O)n1ccnc1)=C(C)N2]

to a compound of formula IIIa

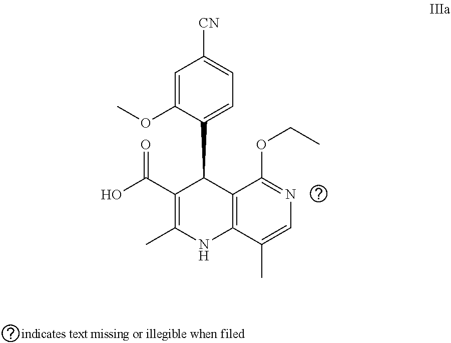 [SMILES: CCOc1ncc(C)c2c1[C@H](c1ccc(C#N)cc1OC)C(C(=O)O)=C(C)N2]

with a chiral acid selected from Di-p-toluoyl-D-tartaric acid or Di-benzoyl-L-tartaric acid. (+)-Di-p-toluoyl-D-tartaric acid (DTTA) of formula

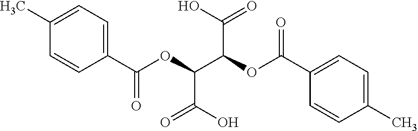

was found to be a suitable chiral acid for the preparation of the (S)-enantiomer of formula IIIa, which can be further converted to (S)-Finerenone.

In a further aspect of the invention, the process according to Scheme 1 involves the preparation of a compound of formula III

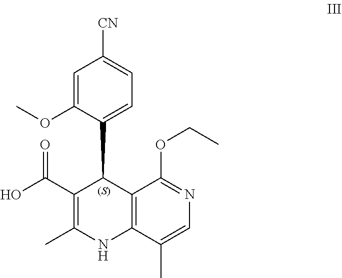

comprising the reaction of a compound of formula II

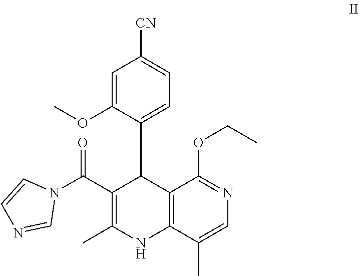 [SMILES: CCOc1ncc(C)c2c1C(c1ccc(C#N)cc1OC)C(C(=O)n1ccnc1)=C(C)N2]

with (+)-Di-p-toluoyl-D-tartaric acid (DTTA) to obtain a compound of formula IIIa

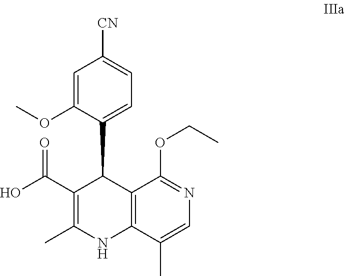 [SMILES: CCOc1ncc(C)c2c1[C@H](c1ccc(C#N)cc1OC)C(C(=O)O)=C(C)N2]

The ratio between compound of formula II to DTTA can be between 1:1 to 1:1.5. Suitable solvents are organic solvents or a mixture of organic solvents. Preferred solvents are acetone or a mixture of 2-Methyl-THF and acetone. 2-Methyl-THF and acetone can be used in a ratio of 1:2 to 1:3, preferred ratio is 1:2.5. The reaction can be performed at a temperature between 20-30 degrees Celsius. Compound of formula IIIa can be isolated by filtration. Compound of formula IIIa can optionally be dried, e.g. under vacuum at a temperature between 50-60 degrees Celsius.

Compound of formula IIIa is then further converted to a compound of formula III by addition of a base in a suitable solvent. Suitable bases are inorganic bases. A preferred base is sodium bicarbonate. A suitable solvent is a mixture of an organic solvent and water, such as 2-Methyl-THF and water. Preferably the ratio between 2-Methyl-THF and water is 1:1. The pH of the reaction mass may be adjusted to a pH between 6.0 to 7.5.

A compound of formula II

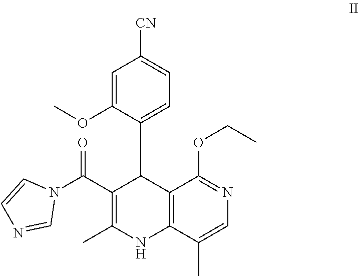 [SMILES: CCOc1ncc(C)c2c1C(c1ccc(C#N)cc1OC)C(C(=O)n1ccnc1)=C(C)N2]

can be prepared from a compound of formula I

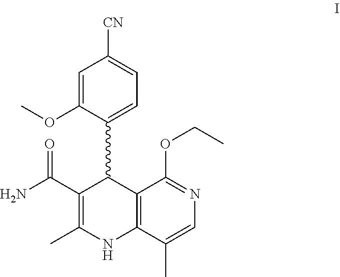 [SMILES: CCOc1ncc(C)c2c1C(c1ccc(C#N)cc1OC)C(C(N)=O)=C(C)N2]

as described, e.g., in U.S. Pat. No. 8,436,180.

The compound of formula III

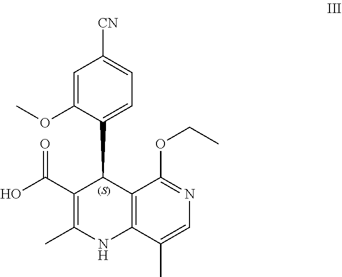

according to the disclosed process is obtained with an enantiomeric excess of around 94% and can be converted to Finerenone without any further purification.

The compound of formula III can be converted to Finerenone by ammonolysis as described, e.g., in U.S. Pat. No. 8,436,180. The carboxylic acid group of compound of formula III can be activated by an activating agent like thionylchloride or 1,1′-carbonyldiimidazole (CDI) in a suitable solvent and then be converted to Finerenone by addition of ammonia.

Optionally, the compound of formula IIIa can be purified prior to the conversion to compound of formula III.

The purification of (S)-4-(4-cyano-2-methoxyphenyl)-5-ethoxy-2, 8,-dimethyl-1, 4-dihydro-1, 6-naphthyridine-3-carboxylic ditoluoyl tartrate salt (compound of formula IIIa) may be performed by using suitable solvents. The suitable solvent may include acetic acid, an alcohol such as methanol, ethanol, 1-propanol, isopropanol, 1-butanol, 2-butanol, 1-pentanol, 1-octanol and the like; a ketone such as acetone, propanone, methylisobutylketone and the like; a nitrile such as acetonitrile, propanenitrile and the like; an ester such as methyl acetate, ethyl acetate, n-propyl acetate, tert-butyl acetate and the like; a haloalkane such as dichloromethane, chloroform and the like; an ether such as dimethyl ether, isopropyl ether, methyl tert-butyl ether and the like; an aromatic hydrocarbon such as toluene and the like; a hydrocarbon such as n-hexane, n-heptane and the like; dimethyl formamide; dimethylsulfoxide; dimethyl acetamide; tetrahydrofuran; N-methylpyrrolidone; water; or mixtures thereof. Preferred solvents include dichloromethane, acetic acid, methanol, ethanol, isopropanol, water and mixture thereof.

The purification of Finerenone, (S)-4-(4-cyano-2-methoxyphenyl-5-ethoxy-2, 8-dimethyl-1, 4-dihydro-1, 6-naphthyridine-3-carboxamide may be performed by using suitable solvents. The suitable solvent may include acetic acid, an alcohol such as methanol, ethanol, 1-propanol, isopropanol, 1-butanol, 2-butanol, 1-pentanol, 1-octanol and the like; a ketone such as acetone, propanone, methylisobutylketone and the like; a nitrile such as acetonitrile, propanenitrile and the like; an ester such as methyl acetate, ethyl acetate, isopropylacetate, n-propyl acetate, tert-butyl acetate and the like; a haloalkane such as dichloromethane, chloroform and the like; an ether such as dimethyl ether, isopropyl ether, methyl tert-butyl ether and the like; an aromatic hydrocarbon such as toluene and the like; a hydrocarbon such as n-hexane, n-heptane and the like; dimethyl formamide; dimethylsulfoxide; dimethyl acetamide; tetrahydrofuran; N-methylpyrrolidone; water; or mixtures thereof. Preferred solvents include acetone, acetic acid, dichloromethane, methanol, ethanol, acetonitrile, DMF, n-hexane and mixture thereof.

In another aspect, the present invention also encompasses a process for the preparation of Finerenone according to Scheme 2.

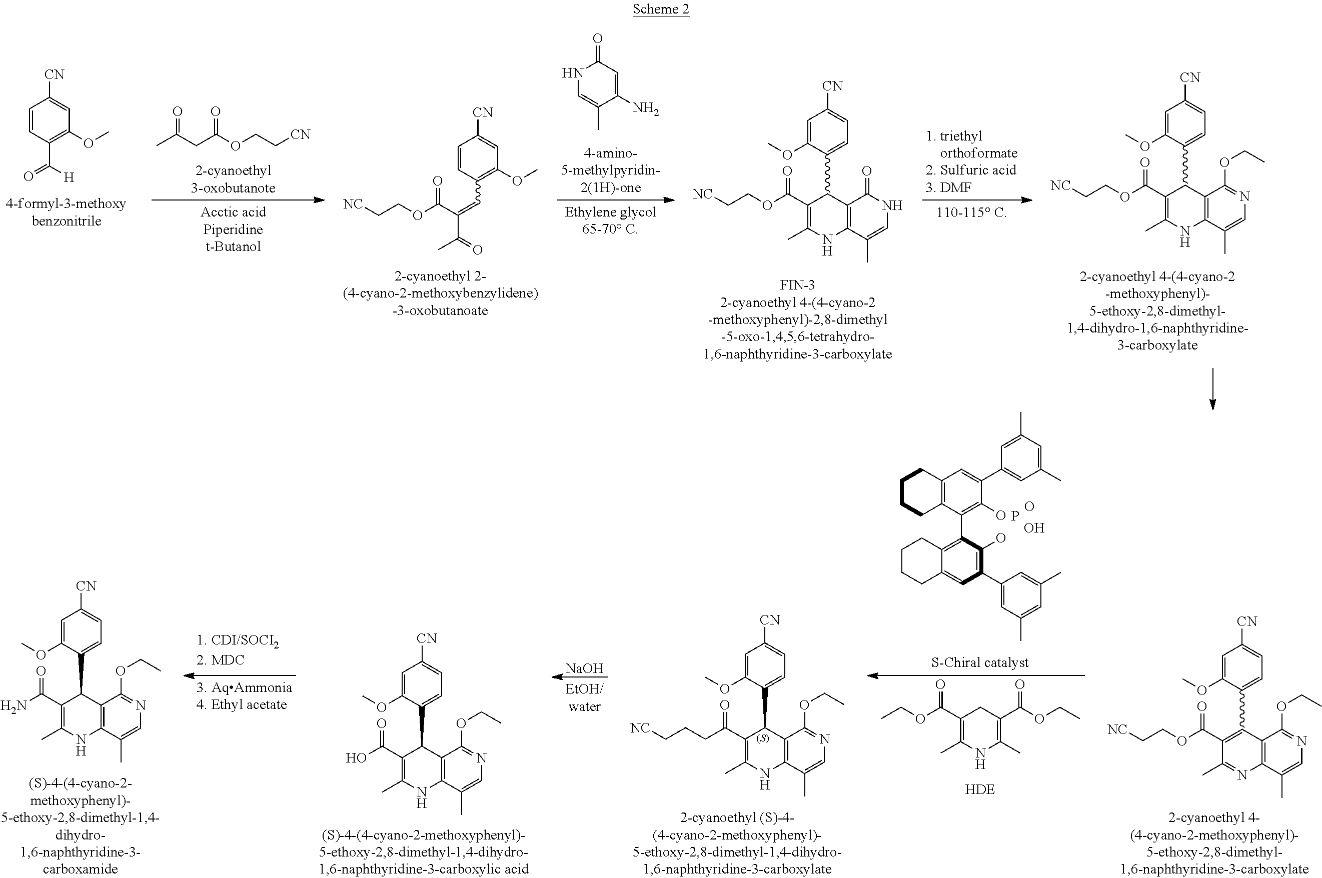

The process according to Scheme 2 involves oxidation of a compound of formula

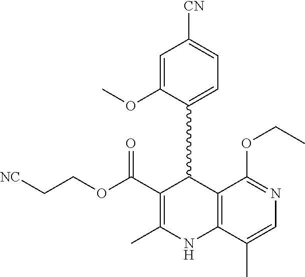 [SMILES: CCOc1ncc(C)c2c1C(c1ccc(C#N)cc1OC)C(C(=O)OCCC#N)=C(C)N2]

to obtain a compound of formula

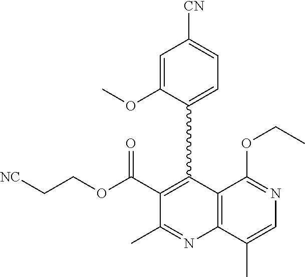 [SMILES: CCOc1ncc(C)c2nc(C)c(C(=O)OCCC#N)c(-c3ccc(C#N)cc3OC)c12]

followed by stereoselective hydrogenation with a chiral catalyst in a suitable solvent to obtain a compound of formula

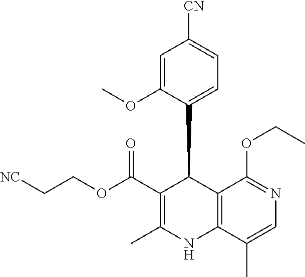 [SMILES: CCOc1ncc(C)c2c1[C@H](c1ccc(C#N)cc1OC)C(C(=O)OCCC#N)=C(C)N2]

which can, optionally, be further converted to (S)-Finerenone.

Stereoselective synthesis of 2-cyanoethyl-(S)-4-(4-cyano-2-methoxyphenyl)-5-ethoxy-2, 8-dimethyl-1, 4-dihydro-1, 6-naphthyridine-3-carboxylate from 2-cyanoethyl4-(4-cyano-2-methoxyphenyl)-5-ethoxy-2,8-dimethyl-1,6-naphthyridine-3-carboxylate may be performed using the chiral phosphoric acid catalyst (S-catalyst) and Hantzsch ester in the presence of a suitable solvent. The suitable solvent may include an alcohol such as methanol, ethanol, 1-propanol, isopropanol, 1-butanol, 2-butanol, 1-pentanol, 1-octanol and the like; a ketone such as acetone, propanone, methylisobutylketone and the like; a nitrile such as acetonitrile, propanenitrile and the like; an ester such as methyl acetate, ethyl acetate, n-propyl acetate, tert-butyl acetate and the like; a haloalkane such as dichloromethane, chloroform and the like; an ether such as dimethyl ether, isopropyl ether, methyl tert-butyl ether and the like; an aromatic hydrocarbon such as toluene and the like; a hydrocarbon such as n-hexane, n-heptane and the like; dimethyl formamide; dimethylsulfoxide; dimethyl acetamide; tetrahydrofuran; N-methylpyrrolidone; water; or mixtures thereof. Preferred solvents include tetrahydrofuran, 2-butanol, methanol, ethanol, 1-butanol, isopropanol and mixture thereof.

The present invention also encompasses a process for preparing(S)-Finerenone as depicted in Scheme 3.

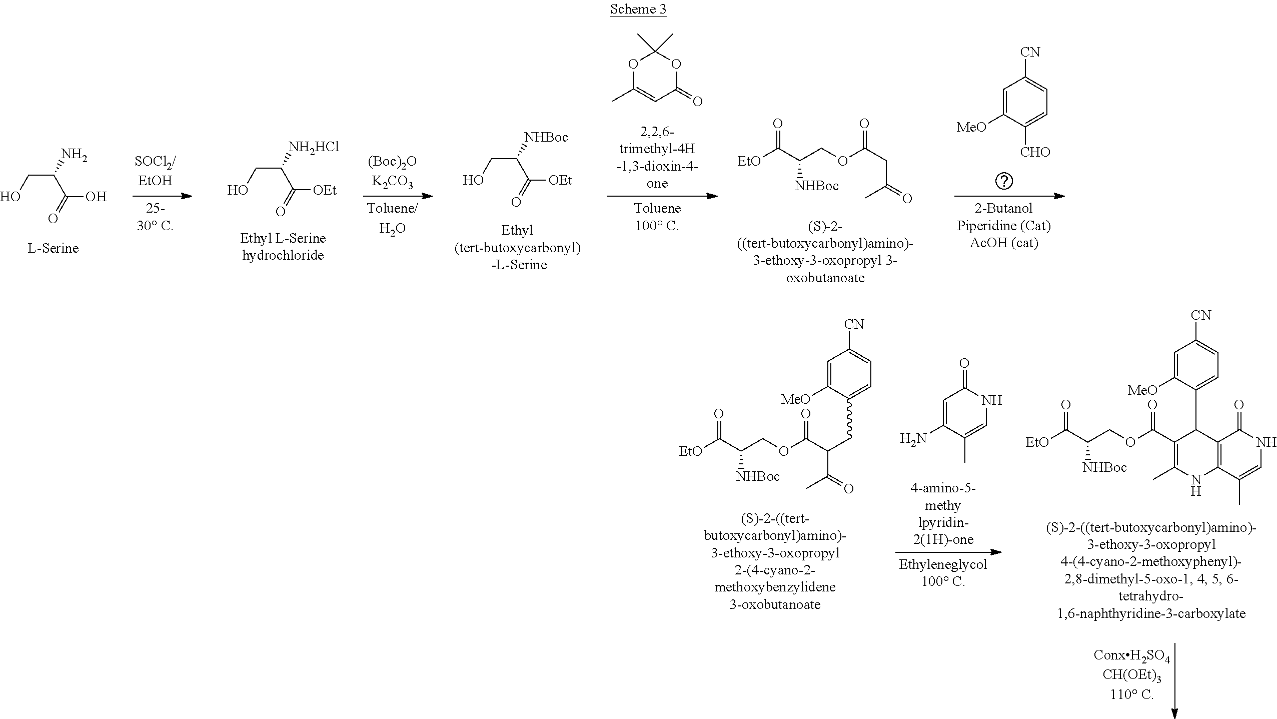

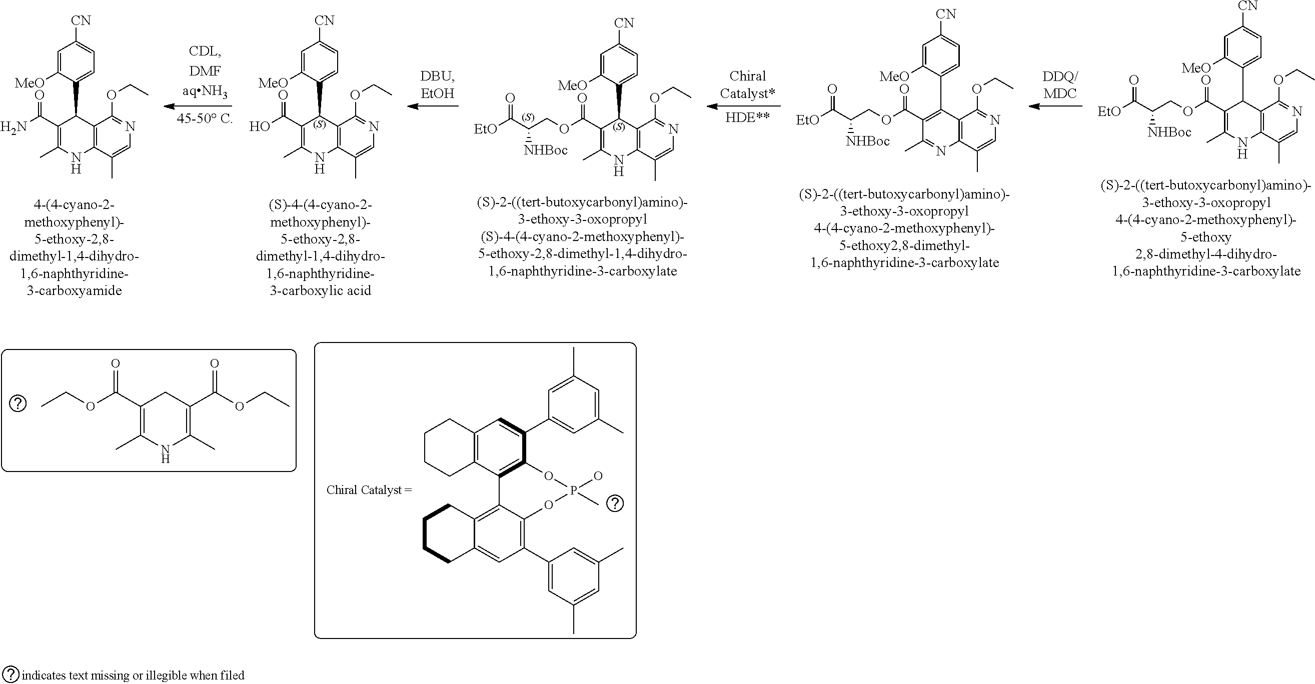

The process according to Scheme 3 involves stereoselective hydrogenation with a chiral catalyst of a compound of formula

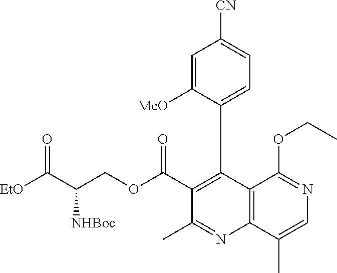 [SMILES: CCOC(=O)[C@H](COC(=O)c1c(C)nc2c(C)cnc(OCC)c2c1-c1ccc(C#N)cc1OC)NC(=O)OC(C)(C)C]

to a compound of formula

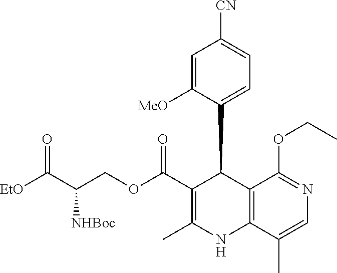 [SMILES: CCOC(=O)[C@H](COC(=O)C1=C(C)Nc2c(C)cnc(OCC)c2[C@@H]1c1ccc(C#N)cc1OC)NC(=O)OC(C)(C)C]

which can be further converted to (S)-Finerenone.

Also provided are certain compounds useful as intermediates for the preparation of (S)-Finerenone. In an additional aspect, the present invention provides the use of these certain compounds in the preparation of (S)-Finerenone.

These compounds can be used in the preparation of (S)-Finerenone.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### EXAMPLES

### Preparation of Finerenone According to Scheme 1

### 1.1 2-cyanoethyl 2-(4-cyano-2-methoxybenzylidene)-3-oxobutanoate

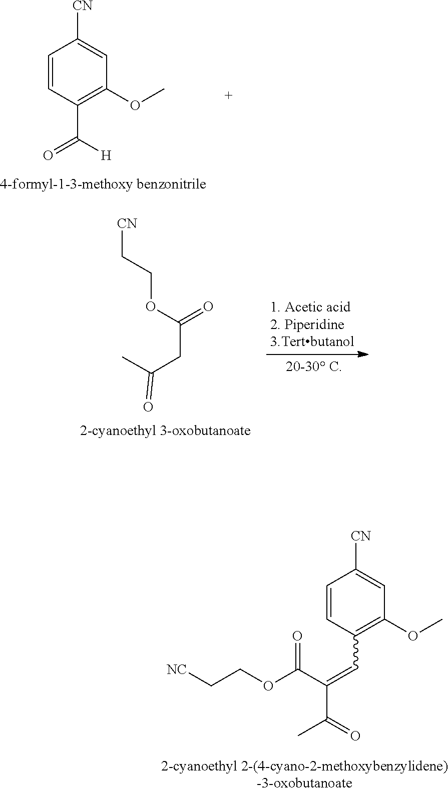 [SMILES: COc1cc(C#N)ccc1C=O ; CC(=O)CC(=O)OCCC#N ; COc1cc(C#N)ccc1C=C(C(C)=O)C(=O)OCCC#N]

Charged 4-Formyl-3-methoxy benzonitrile (100 grams, 0.620 mmol), 2-cyanomethyl-3-oxobutanoate (129.96 grams, 0.837 mmol), acetic acid 3.72 grams, 0.062 mmol) Piperidine (5.30 grams, 0.062 mmol) and tert. Butanol (500 ml) into the flask. Reaction mass stirred for 10 to 14 hours at 20-30° C. Reaction was monitored by HPLC. After reaction completion, filtered and washed with IPA (200 ml). Wet material dried under vacuum at 55-65° C. to yield 2-cyanoethyl 2-(4-cyano-2-methoxybenzylidene)-3-oxobutanoate (177 grams, Purity=96.02%, Yield≈95%).

¹H-NMR: 2.47 (s, 3H), 2.69 (t, 2H, J=6.15 Hz), 3.92 (s, 3H), 4.39 (t, 2H, J=6.14), 7.17 (s, 1H), 7.27 (D, 1H, J=7.93 Hz), 7.45 (d, 1H, J=7.90), 7.84 (s, 1H). [M+H]⁺: 299.10.

### 1.2 2-cyanoethyl 4-(4-cyano-2-methoxyphenyl)-2, 8,-dimethyl-5-oxo-1, 4, 5, 6-tetrahydro-1 6-naphthyridine-3-carboxylate

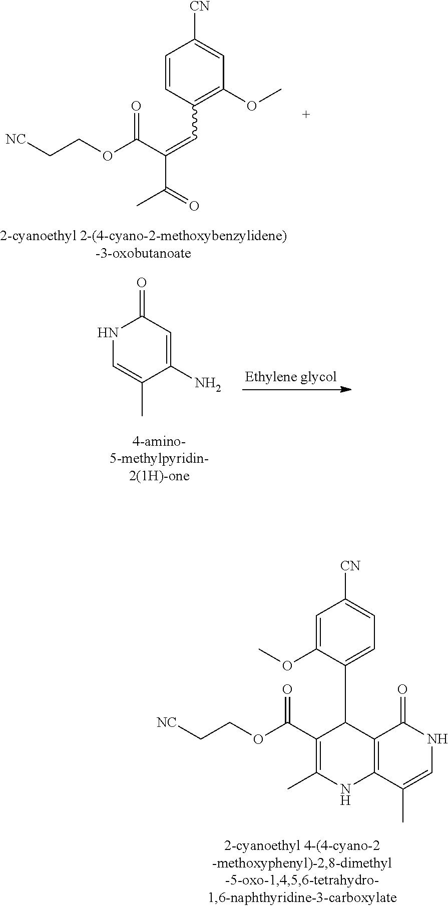

Charged 2-cyanomethyl-2-(4-cyano-2-methoxybenzylidene-3-oxobutanoate (100 grams, 0.335 mmol), 4-amino-5-methyl-pyridin-2 (1H)-one (39.5 grams, 0.318 mmol) and Diethylene glycol (200 ml) into the flask. Reaction mass heated to 85-95° for 6 to 12 hours and reaction progress was monitored by HPLC. After reaction completion, cooled to 20-30° C. followed by IPA (200 ml) added. The reaction mass further cooled to 0-5° C. and stirred for 1.0-2.0 hours at 0-5° C., then filtered, washed with IPA (100 ml). Wet material dried under vacuum at 65-75° C. to provided 2-cyanoethyl 4-(4-cyano-2-methoxyphenyl)-2, 8,-dimethyl-5-oxo-1, 4, 5, 6-tetrahydro-1, 6-naphthyridine-3-carboxylate (79 grams, Purity=97%, Yield≈58%).

¹H-NMR: 2.03 (s, 3H), 2.36 (s, 3H), 2.80 (m, 2H), 3.75 (s, 3H), 4.04 (m, 1H), 4.11 (m, 1H), 5.23 (s, 1H), 6.85 (s, 1H), 7.25 (m, 1H), 7.31 (m, 2H), 8.19 (s, 1H), 10.78 (s, 1H); [M+H]⁺: 405.17).

### 1.3 2-cyanoethyl 4-(4-cyano-2-methoxyphenyl)-5-ethoxy-2, 8,-dimethyl-1, 4-dihydro-1, 6-naphthyridine-3-carboxylate

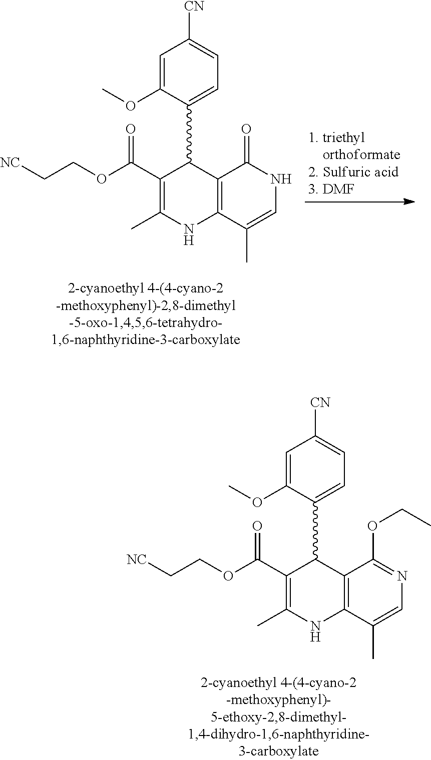 [SMILES: COc1cc(C#N)ccc1C1C(C(=O)OCCC#N)=C(C)Nc2c(C)c[nH]c(=O)c21 ; CCOc1ncc(C)c2c1C(c1ccc(C#N)cc1OC)C(C(=O)OCCC#N)=C(C)N2]

Charged 2-cyanoethyl 4-(4-cyano-2-methoxyphenyl)-2, 8,-dimethyl-5-oxo-1, 4, 5, 6-tetrahydro-1, 6-naphthyridine-3-carboxylate (50 grams, 0.123m mol), triethyl ortho formate (200 ml), DMF (100 ml) followed by con. sulphuric acid (1.21 grams, 0.012 mmol) into the flask. Reaction mass heated to 105-110° C. for 6 to 12 hours. The reaction progress monitored by HPLC. After reaction completion, cooled to 5-15° C. and water (300 ml) added, stirred for 2 hours at 5-15° C., filtered and washed with water (50 ml), wet material dried under vacuum at 55-65° C. to provided 2-cyanoethyl 4-(4-cyano-2-methoxyphenyl)-5-ethoxy-2, 8,-dimethyl-1, 4-dihydro-1, 6-naphthyridine-3-carboxylate, (45 grams, Purity=97%, Yield≈85%).

¹H-NMR: 1.22 (t, 3H, J=7.05), 2.16 (s, 3H), 2.47 (s, 3H), 2.63 (m, 2H), 3.79 (s, 3H), 4.16 (q, 2H, J=7.51), 4.22 (m, 2H), 5.48 (s, 1H), 6.13 (s, 1H), 7.04 (d, 1H, J=1.14), 7.16 (dd, 1H, J=8.48, 0.69), 7.42 (d, 1H, J=7.81), 7.65 (s, 1H); [M+H]⁺: 433.17.

### 1.4 4-(4-cyano-2-methoxyphenyl)-5-ethoxy 2, 8,-dimethyl-1, 4-dihydro-1, 6-naphthyridine-3-carboxylic Acid

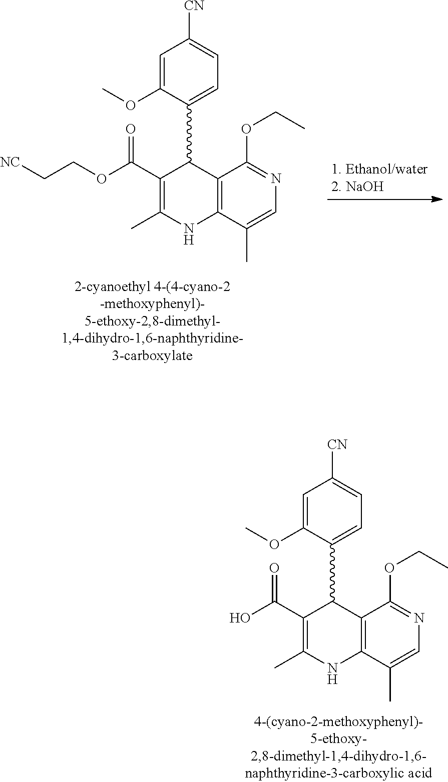

Charged 2-cyanoethyl 4-(4-cyano-2-methoxyphenyl)-5-ethoxy-2, 8,-dimethyl-1, 4-dihydro-1, 6-naphthyridine-3-carboxylate (30 grams, 0.069 mmol), 75 mL ethanol and 30 mL water. To the reaction mass, Aq. Sodium hydroxide solution (4.16 grams, 0.104 mmol dissolved in 45 mL water) slowly added for 30 to 45 minutes and the reaction was monitored by HPLC. After reaction completion, extracted with toluene (60 ml) and Aq. Layer separated. Aq. Layer was adjusted to pH 6.5 by using 50% aq. solution of acetic acid. 4-(4-cyano-2-methoxyphenyl)-5-ethoxy 2, 8,-dimethyl-1, 4-dihydro-1, 6-naphthyridine-3-carboxylic acid solid precipitated out and stirred for 1-2 hours at 20-30° C., filtered and washed with water (30 mL), dried under vacuum at 45-55° C. to provided 4-(4-cyano-2-methoxyphenyl)-5-ethoxy 2, 8,-dimethyl-1, 4-dihydro-1, 6-naphthyridine-3-carboxylic acid, (26 grams, Purity=92%, Yield≈98%).

¹H-NMR: 1.12 (t, 3H, J=6.97), 2.39 (s, 3H), 2.15 (s, 3H), 3.76 (s, 3H), 4.05 (m, 2H), 5.35 (s, 1H), 7.28 (m, 1H), 7.28 (m, 1H), 7.32 (s, 1H), 7.57 (s, 1H), 8.15 (s, 1H), 11.44 (s, 1H). Mass: [M+H]⁺: 380.15.

### 1.5 2-cyanoethyl 4-(4-cyano-2-methoxyphenyl)-5-ethoxy-2, 8,-dimethyl-1, 4-dihydro-1, 6-naphthyridine-3-carboxylate

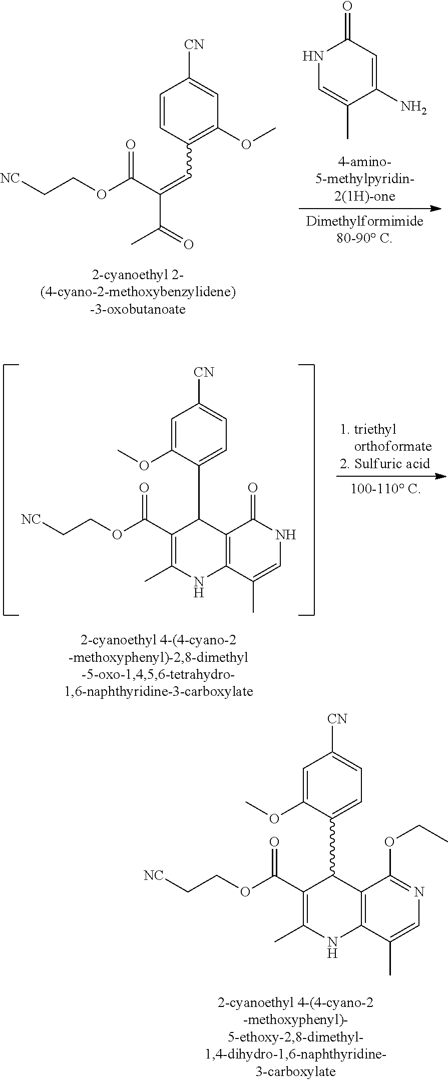

Charged 2-cyanoethyl-2-(4-cyano-2-methoxybenzylidene-3-oxobutanoate (5.0 grams, 0.016 mmol), 4-amino-5-methyl-pyridin-2 (1H)-one (2.0 grams, 0.016 mmol) and Dimethylformamide (10 ml) into the flask. Reaction mass heated to 85-95° for 6 to 12 h and reaction progress was monitored by HPLC. After reaction completion, cooled to 20-30° C., added triethyl orthoformate (20 ml) followed by con. sulphuric acid (0.32 grams, 0.0032 mol) into the flask. Reaction mass heated to 105-110° C. for 6 to 12 hours. The reaction progress monitored by HPLC. After reaction completion, cooled to 20-30° C. and water (50 ml) and Ethyl acetate (50 ml), stirred for 30 minutes. Layers were separated organic layers distilled to afford 5.5 grams of 2-cyanoethyl 4-(4-cyano-2-methoxyphenyl)-5-ethoxy-2, 8,-dimethyl-1, 4-dihydro-1, 6-naphthyridine-3-carboxylate as oily mass (wt 5.5 grams).

### 1.6 4-(5-ethoxy-3-(1H-imidazole-1-carbonyl)-2, 8,-dimethyl-1, 4-dihydro-1, 6-naphthyridi-4yl)-3-methoxybenzonitrile

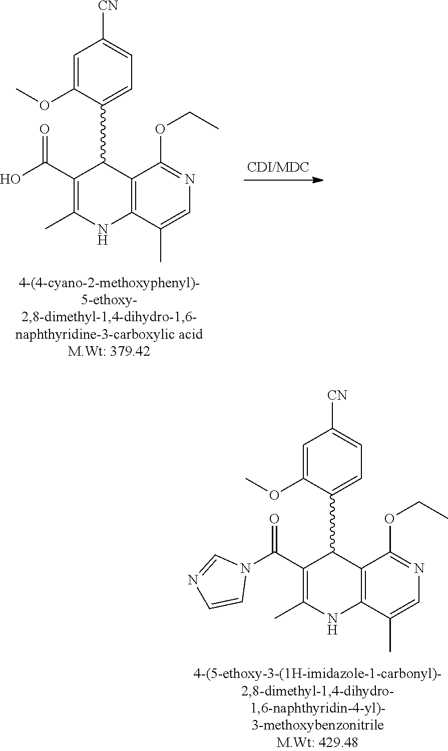 [SMILES: CCOc1ncc(C)c2c1C(c1ccc(C#N)cc1OC)C(C(=O)O)=C(C)N2 ; CCOc1ncc(C)c2c1C(c1ccc(C#N)cc1OC)C(C(=O)n1ccnc1)=C(C)N2]

Charged 4-(4-cyano-2-methoxyphenyl)-5-ethoxy 2, 8,-dimethyl-1, 4-dihydro-1, 6-naphthyridine-3-carboxylic acid (25 g 0.065 mmol), 1, 1′-Carbonyldiimidazole (CDI) (16 grams, 0.0986 mmol) followed by Dichloromethane (125 ml) added and stirred for 2-4 hours. Reaction was monitored by HPLC. After reaction completion, water added (125 volume) then organic layer separated out. Organic layer distilled out till 0.25 volume MDC inside the flask and stripped out with heptane (50 ml), finally heptane (250 ml) added and stirred for 1-2 hours then filtered. Material dried under vacuum at 40-50° C. to provide 4-(5-ethoxy-3-(1H-imidazole-1-carbonyl)-2, 8,-dimethyl-1, 4-dihydro-1, 6-naphthyridin-4yl)-3-methoxybenzonitrile (27 grams, Purity=96%, Yield≈96%).

¹H-NMR: 1.10 (m, 3H), 2.05 (s, 3H), 2.23 (s, 3H), 3.61 (s, 3H), 4.14 (m, 2H), 5.62 (s, 1H), 6.27 (s, 1H), 6.80 (d, 1H), 6.98 (s, 1H), 7.09 (s, 2H), 7.37 (s, 1H), 7.79 (s, 1H), 7.97 (s, 1H).

### 1.7 (S)-4-(4-cyano-2-methoxyphenyl)-5-ethoxy-2, 8,-dimethyl-1, 4-dihydro-1, 6-Naphthyridine-3-carboxylic Ditoluoyl Tartrate Salt

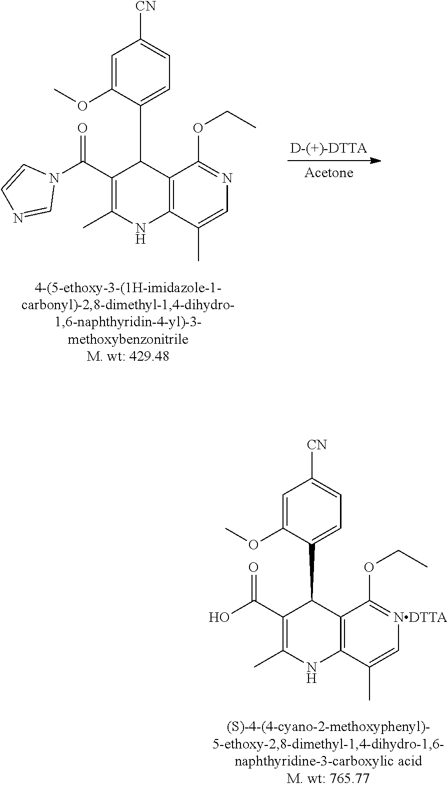

Charged 4-(5-ethoxy-3-(1H-imidazole-1-carbonyl)-2, 8,-dimethyl-1, 4-dihydro-1, 6-naphthyridi-4yl)-3-methoxybenzonitrile (5.0 g 0.011 mmol), (+)-Di-p-toluyl-D-tartaric acid (5.39 grams, 0.0139 mmol) and acetone (20 ml). Stirred for 12-15 hours at 20-30° C. Filtered and washed with acetone (5 ml). Material dried under vacuum at 55-65° C. to provide(S)-4-(4-cyano-2-methoxyphenyl)-5-ethoxy-2, 8,-dimethyl-1, 4-dihydro-1, 6-Naphthyridine-3-carboxylic di-toluyl tartrate salt (5.0 grams, chiral Purity=94%, Yield≈52.9%).

### 1.8 Purification of (S)-4-(4-cyano-2-methoxyphenyl)-5-ethoxy-2, 8,-dimethyl-1, 4-dihydro-1, 6-naphthyridine-3-carboxylic Ditoluoyl Tartrate Salt

Charged (S)-4-(4-cyano-2-methoxyphenyl)-5-ethoxy-2, 8,-dimethyl-1, 4-dihydro-1, 6-naphthyridine-3-carboxylic ditoluoyl tartrate salt (0.5 grams, 0.0006 mmol), water/acetic acid (50:50 mix) (5 ml) into the flask. Heated to 50-60° C. for 1-2 hours. Cooled to 20-30° C. and stirred for 1-2 hours. Filtered and washed with acetic acid (0.5 ml). Material dried under vacuum at 50-60° C. to provide(S)-4-(4-cyano-2-methoxyphenyl)-5-ethoxy-2, 8,-dimethyl-1, 4-dihydro-1, 6-naphthyridine-3-carboxylic ditoluoyl tartrate salt. (0.1 g, Yield≈25%, chiral purity=98%).

### 1.9 Purification of (S)-4-(4-cyano-2-methoxyphenyl)-5-ethoxy-2, 8,-dimethyl-1, 4-dihydro-1, 6-naphthyridine-3-carboxylic Ditoluoyl Tartrate Salt

Charged (S)-4-(4-cyano-2-methoxyphenyl)-5-ethoxy-2, 8,-dimethyl-1, 4-dihydro-1, 6-naphthyridine-3-carboxylic ditoluoyl tartrate salt (0.1 g 0.0001 mmol), MDC/Methanol (50:50) mixture (0.50 ml) into the flask. Heated to 50-60° C. for 1-2 hours. Cooled to 20-30° C. and stirred for 1-2 hours. Filtered and washed with acetic acid (0.5 ml). Material dried under vacuum at 50-60° C. to provide(S)-4-(4-cyano-2-methoxyphenyl)-5-ethoxy-2, 8,-dimethyl-1, 4-dihydro-1, 6-naphthyridine-3-carboxylic ditoluoyl tartrate salt. (0.06 grams, Yield≈25%, chiral purity >99%).

### 1.10 Purification of (S)-4-(4-cyano-2-methoxyphenyl)-5-ethoxy-2, 8,-dimethyl-1, 4-dihydro-1, 6-naphthyridine-3-carboxylic Ditoluoyl Tartrate Salt

Charged (S)-4-(4-cyano-2-methoxyphenyl)-5-ethoxy-2, 8,-dimethyl-1, 4-dihydro-1, 6-naphthyridine-3-carboxylic ditoluoyl tartrate salt (0.1 grams, 0.0001 mmol), Methanol/acetic acid (50:50) mixture (0.50 ml) into the flask. Heated to 50-60° C. for 1-2 hours. Cooled to 20-30° C. and stirred for 1-2 hours. Filtered and washed with same solvent mixture (0.1 ml). Material dried under vacuum at 50-60° C. to provide(S)-4-(4-cyano-2-methoxyphenyl)-5-ethoxy-2, 8,-dimethyl-1, 4-dihydro-1, 6-naphthyridine-3-carboxylic ditoluoyl tartrate salt. (0.06 grams, Yield ≈25%, chiral purity >98%).

### 1.11 (S)-4-(4-cyano-2-methoxyphenyl)-5-ethoxy 2, 8,-dimethyl-1, 4-dihydro-1, 6-naphthyridine-3-carboxylic Acid

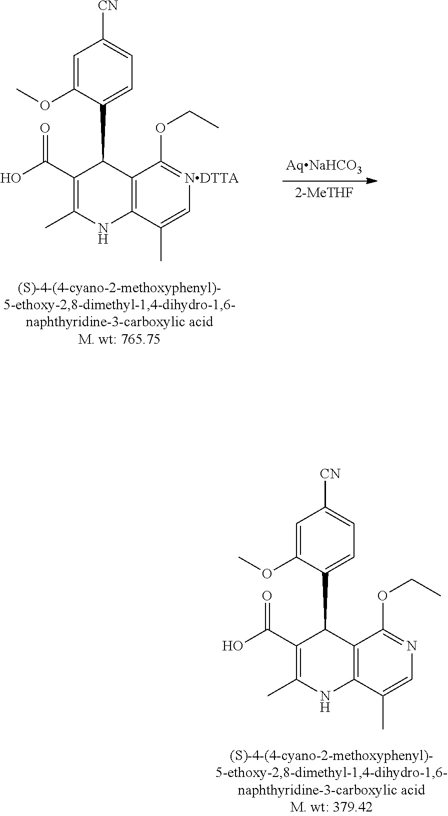

Charged (S)-4-(4-cyano-2-methoxyphenyl)-5-ethoxy-2, 8,-dimethyl-1, 4-dihydro-1, 6-naphthyridine-3-carboxylic ditoluoyl tartrate salt (7.0 grams, 0.0091 mmol), and 2-MeTHF (70 ml) and water (70 ml) into the flask. Reaction mass pH adjusted to 6 to 6.5 by using 2% Aq. Sodium bicarbonate solution. Stirred for 15-30 min and organic layer separated and concentrated to provide(S)-4-(4-cyano-2-methoxyphenyl)-5-ethoxy 2, 8,-dimethyl-1, 4-dihydro-1, 6-naphthyridine-3-carboxylic acid (3.2 g, yield: 94.1% chiral purity: 94%).

¹H-NMR: 1.12 (t, 3H, J=6.97), 2.39 (s, 3H), 2.15 (s, 3H), 3.76 (s, 3H), 4.05 (m, 2H), 5.35 (s, 1H), 7.28 (m, 1H), 7.28 (m, 1H), 7.32 (s, 1H), 7.57 (s, 1H), 8.15 (s, 1H), 11.44 (s, 1H); Mass: 380.15 (M+H).

### 1.12 (S)-4-(4-cyano-2-methoxyphenyl-5-ethoxy-2, 8-dimethyl-1, 4-dihydro-1, 6-naphthyridine-3-carboxamide

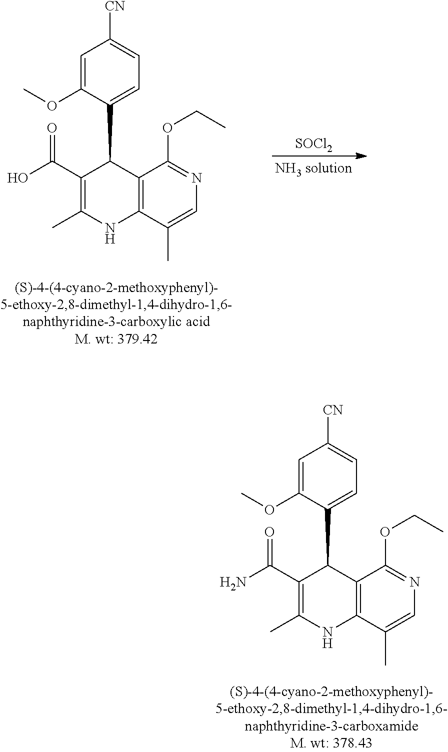

Charged (S)-4-(4-cyano-2-methoxyphenyl)-5-ethoxy 2, 8,-dimethyl-1, 4-dihydro-1, 6-naphthyridine-3-carboxylic acid (2.50 grams, 0.006 mmol), MDC (25 ml) into the flask and slowly Thionylchloride (1.17 grams, 0.0098 mmol) added at 5-10° C. for 30 min. reaction mass stirred for 1-2 at 20-30° C. The above reaction mass quenched into Aq. Ammonia solution (50 ml) at 20-30° C. After reaction completion, organic layer separated and washed with water (2.50 ml), organic layer concentrated and added ethyl acetate (12.5 ml). Heated to 50-60° C. for 1 h and cooled to 20-30° C. Stirred for 1-2 hours. Filtered to provide(S)-4-(4-cyano-2-methoxyphenyl-5-ethoxy-2, 8-dimethyl-1, 4-dihydro-1, 6-naphthyridine-3-carboxamide (2 grams, Purity=71%, Yield≈50-55%).

¹H-NMR: 1.06 (t, 3H, J=6.50), 2.13 (s, 3H), 2.20 (s, 3H), 3.83 (s, 3H), 4.02 (m, 2H), 5.39 (s, 1H), 6.79 (m, 2H), 7.16 (d, 1H, J=7.36), 7.29 (d, 1H, J=7.16), 7.38 (s, 1H), 7.56 (s, 1H), 7.71 (s, 1H). Mass: [M+H]⁺: 379.17.

### 1.13 Purification of (S)-4-(4-cyano-2-methoxyphenyl-5-ethoxy-2, 8-dimethyl-1, 4-dihydro-1, 6-naphthyridine-3-carboxamide

Charged (S)-4-(4-cyano-2-methoxyphenyl-5-ethoxy-2, 8-dimethyl-1, 4-dihydro-1, 6-naphthyridine-3-carboxamide crude (0.10 grams, 0.0002 mmol), acetone (0.10 ml) into the flask and heated to 40-50° C. for 1-2h. Cooled to 20-30° C. and stirred for 1-2h. Filtered, washed with acetone (0.10 ml). Dried under vacuum at 50-60° C. to provide title compound (0.05 grams, Purity=98%, Yield≈45-50%).

### 1.14 Purification of (S)-4-(4-cyano-2-methoxyphenyl-5-ethoxy-2, 8-dimethyl-1, 4-dihydro-1, 6-naphthyridine-3-carboxamide

Charged (S)-4-(4-cyano-2-methoxyphenyl-5-ethoxy-2, 8-dimethyl-1, 4-dihydro-1, 6-naphthyridine-3-carboxamide crude (0.10 grams, 0.0002 mmol), dichloromethane (2.5 ml) into the flask and heated to 40-50° C. for 1-2 hours and add n-hexane (1.25 ml) then reaction mass cooled to 20-30° C. and stirred for 1-2 hours. Filtered, washed with dichloromethane and n-hexane mixture (0.50 ml). Dried under vacuum at 50-60° C. to provide title compound (0.06 grams, Purity=>98%, Yield: 60%).

### 1.15 Purification of (S)-4-(4-cyano-2-methoxyphenyl-5-ethoxy-2, 8-dimethyl-1, 4-dihydro-1, 6-naphthyridine-3-carboxamide

Charged (S)-4-(4-cyano-2-methoxyphenyl-5-ethoxy-2, 8-dimethyl-1, 4-dihydro-1, 6-naphthyridine-3-carboxamide crude (0.10 grams, 0.0002 mmol), Acetonitrile (1.0 ml) into the flask and heated to 40-50° C. for 1-2 hours, then reaction mass cooled to 20-30° C. and stirred for 1-2h. Filtered, washed with acetonitrile (0.50 ml). Dried under vacuum at 50-60° C. to provide title compound (0.04 grams, Purity=>98%, Yield: 40%).

### 1.16 Purification of (S)-4-(4-cyano-2-methoxyphenyl-5-ethoxy-2, 8-dimethyl-1, 4-dihydro-1, 6-naphthyridine-3-carboxamide

Charged (S)-4-(4-cyano-2-methoxyphenyl-5-ethoxy-2, 8-dimethyl-1, 4-dihydro-1, 6-naphthyridine-3-carboxamide crude (0.10 grams, 0.0002 mmol), Dimethylformamide (1.0 ml) into the flask and heated to 40-50° C. for 1-2h then reaction mass cooled to 20-30° C. and stirred for 1-2h. Filtered, washed with Dimethylformamide (0.50 ml). Dried under vacuum at 50-60° C. to provide title compound (0.05 grams, Purity=>98%, Yield: 50%).

### Preparation of Finerenone According to Scheme 2

### 2.1 2-cyanoethyl-4-(4-cyano-2-methoxyphenyl)-5-ethoxy-2, 8-dimethyl-1, 6-naphthyridine-3-carboxylate

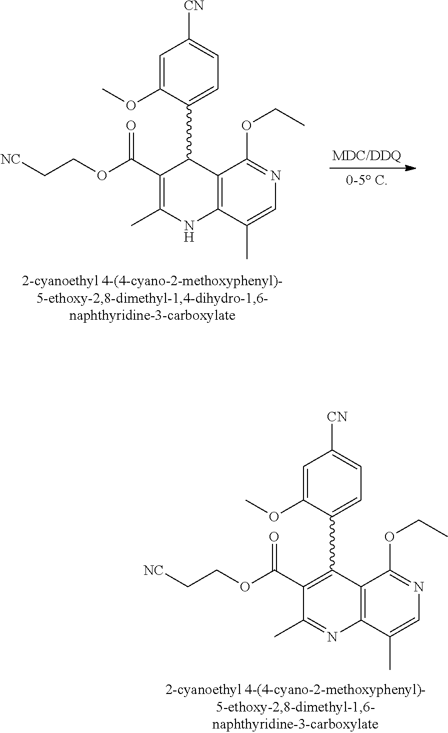 [SMILES: CCOc1ncc(C)c2c1C(c1ccc(C#N)cc1OC)C(C(=O)OCCC#N)=C(C)N2 ; CCOc1ncc(C)c2nc(C)c(C(=O)OCCC#N)c(-c3ccc(C#N)cc3OC)c12]

2-cyanoethyl4-(4-cyano-2-methoxyphenyl)-5-ethoxy-2,8-dimethyl-1,4-dihydro-1,6-naphthyridine-3-carboxylate (10.00 grams, 0.023 mmol) dissolved in MDC (100 ml). 4,5-dichloro-3,6-dioxocyclohexa-1,4-diene-1,2-dicarbonitrile (6.28 grams, 0.027 mmol) added to reaction mass at 0-5° C. The reaction was stirred at 0-5° C. for 2-4 hours. After completion of reaction mass filtered. Residue washed with MDC (50 mL). Filtrate distilled off under vacuum and residue treated with ethanol (50 mL). Solid filtered and washed with 20 mL ethanol to provide 2-cyanoethyl-4-(4-cyano-2-methoxyphenyl)-5-ethoxy-2, 8-dimethyl-1, 6-naphthyridine-3-carboxylate. (8.80 grams, Yield: 88.45%).

¹H-NMR: 0.80 (t, 3H), 2.38 (m, 1H), 2.53 (m, 1H), 2.59 (s, 3H), 2.79 (s, 3H), 3.75 (s, 3H), 4.06 (m, 1H), 4.19 (m, 3H), 7.19 (m, 2H), 7.34 (d, 1H), 8.06 (s, 1H). Mass: [M+H]⁺: 431.19.

### 2.2 2-cyanoethyl-(S)-4-(4-cyano-2-methoxyphenyl)-5-ethoxy-2, 8-dimethyl-1, 4-dihydro-1, 6-naphthyridine-3-carboxylate

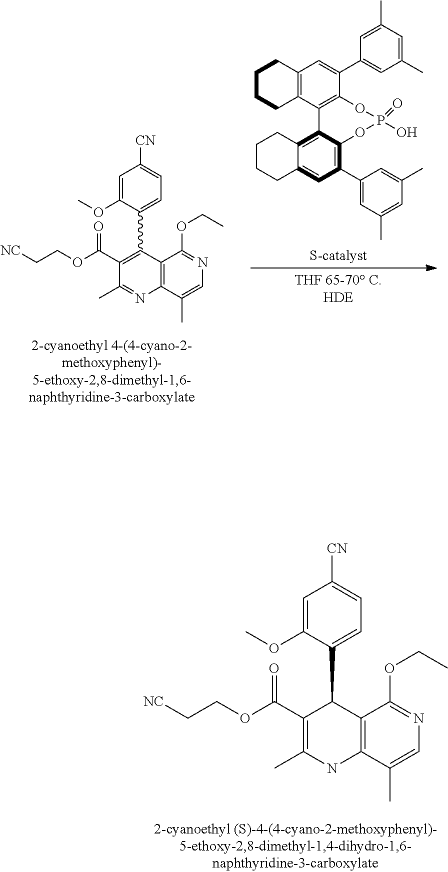

2-cyanoethyl4-(4-cyano-2-methoxyphenyl)-5-ethoxy-2,8-dimethyl-1,6-naphthyridine-3-carboxyl-ate (1.0 grams, 0.0023 mmol) the Hantzsch ester (2.30 grams, 0.000920 mmol) and the chiral phosphoric acid catalyst (S-catalyst) (10 mol %) were added to single neck RBF with 10 mL THF. The reaction was stirred at 65-70° C. in an oil bath for 35-40 hours. Then, the reaction was cooled to room temperature. The organic solvents were evaporated and finally, the crude material was purified by recrystallization from MDC/Heptane (1:3) to provide 2-cyanoethyl-(S)-4-(4-cyano-2-methoxyphenyl)-5-ethoxy-2, 8-dimethyl-1, 4-dihydro-1, 6-naphthyridine-3-carboxylate. 0.70 grams, yield: 70.00%.

### 2.3 2-cyanoethyl-(S)-4-(4-cyano-2-methoxyphenyl)-5-ethoxy-2, 8-dimethyl-1, 4-dihydro-1, 6-naphthyridine-3-carboxylate

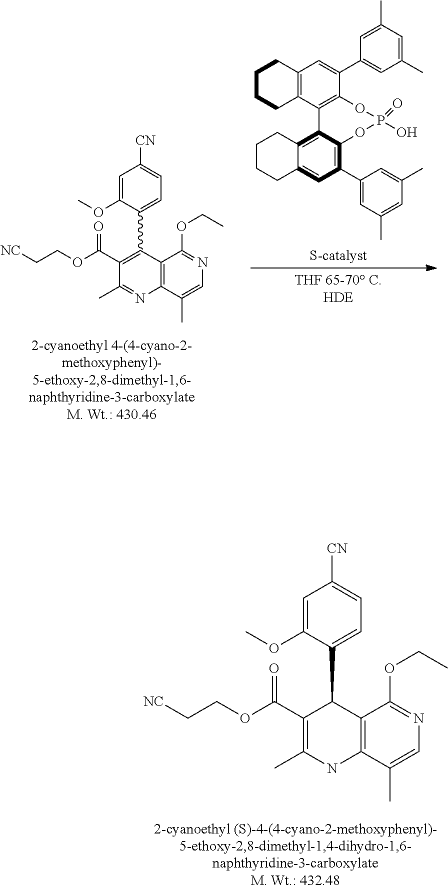

2-cyanoethyl4-(4-cyano-2-methoxyphenyl)-5-ethoxy-2,8-dimethyl-1,6-naphthyridine-3-carboxylate (1.0 grams, 0.0023 mmol)) and the chiral phosphoric acid catalyst (S-catalyst) (10 mol %), and the Hantzsch ester (2.35 grams, 0.009 mmol were added lot wise in 2-butanol (10 vol) to single neck RBF. The reaction was stirred at 95-100° C. in an oil bath for 12-15 hours. Then, the reaction was cooled to room temperature to afford 2-cyanoethyl-(S)-4-(4-cyano-2-methoxyphenyl)-5-ethoxy-2, 8-dimethyl-1, 4-dihydro-1, 6-naphthyridine-3-carboxylate (0.7 grams, yield: 70.00%).

### Preparation of Finerenone According to Scheme 3

### 3.1 Ethyl L-Serinate Hydrochloride

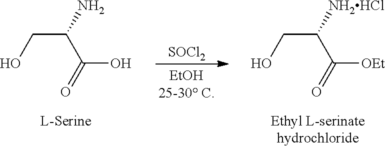

L-Serine (25.0 grams, 0.237 mmol) and methanol (150 ml) charged into a 500 ml round bottom flask. Reaction mass cool down to 0-5° C. and dropwise thionyl chloride (32.8 grams, 0.275 mmol) charged into the reaction mixture upon stirring (exothermic addition). After complete addition temperature rise to 25-30° C. and stirred for 15 hours at this temperature. After completion of the reaction methanol distilled out and 50 ml ethyl acetate charged and stirred for 15 minutes. Solid precipitate filtered out to obtain white solid (32.0 grams, Yield=79%, Mass: [M+H]⁺: 170.70, which directly converted to next step without further purification.

### 3.2 Ethyl (Tert-Butoxycarbonyl)-L-Serinate

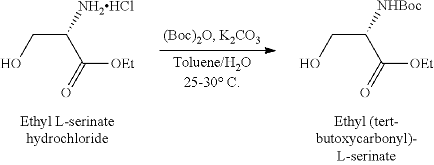

Ethyl L-serinate hydrochloride (30.0 grams, 0.192 mmol) and toluene (150 ml) charged into a 500 ml round bottom flask. Reaction mass cool down to 0-5° C.; charged K₂CO₃ (27.9 grams, 0.202 mmol) and stirred for 10 minutes. Charged Boc-anhydride (46.2 grams, 0.212 mmol) dropwise and after complete addition raise temperature to 25-30° C. and reaction mass stirred for 4 hrs. After completion of the reaction layer separated and toluene layer collected; evaporated to dryness to obtain oily mass. Charged heptane (60 ml) and stirred for 2 hours, in which free flowing solid was obtained. After filtration white solid of Ethyl (tert-butoxycarbonyl)-L-serinate is obtained (33.0 grams, Yield=80%, Mass: [M+H]⁺: 234.28 which directly converted to next step without further purification.

### 3.3 (S)-2-((tert-butoxycarbonyl)amino)-3-ethoxy-3-oxopropyl-3-oxobutanoate

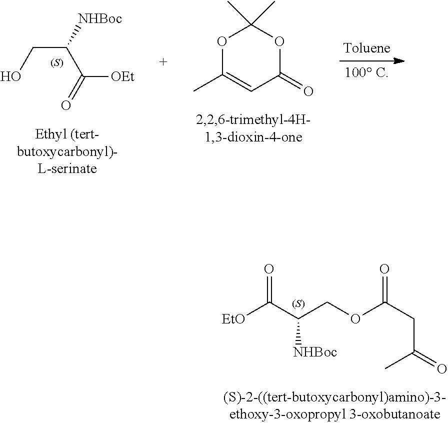

Ethyl (tert-butoxycarbonyl)-L-serinate (30.0 grams, 0.128 mmol) and 2,2,6-trimethyl-4H-1,3-dioxin-4-one (18.2 grams, 0.128 mmol) charged in a 250 ml round bottom flask and stirred for 4 hours at 100-105° C. After reaction completion, reaction mass was used directly for next step without further purification.

### 3.4 (S)-2-((tert-butoxycarbonyl) amino-3-ethoxy-3-oxopropyl-2-(4-cyano-2-methoxybenzylidine)-3-oxobutaboate

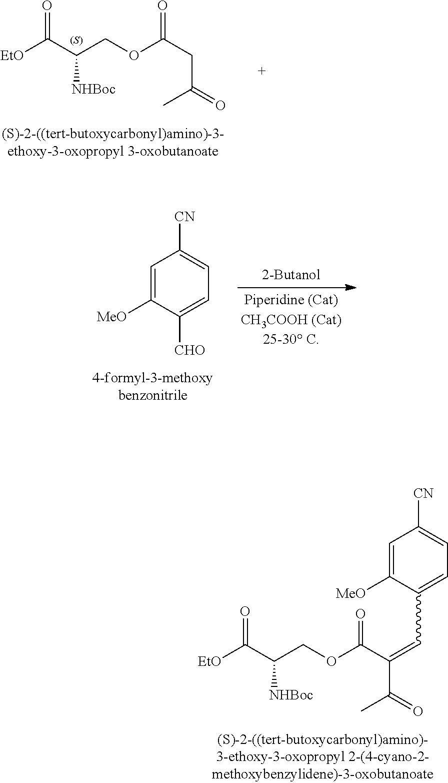

250 ml round bottom flask charged with(S)-2-((tert-butoxycarbonyl)amino)-3-ethoxy-3-oxopropyl 3-oxobutanoate (30.0 grams, 0.094 mmol) and 4-formyl-3-methoxybenzonitrile (15.23 grams, 0.094 mmol) in 2-Butanol (150 ml). Charged piperidine (0.80 grams, 0.009 mmol) followed by acetic acid (0.54 grams, 0.009 mmol) in the reaction mixture and stirred for 6-8 hours at 25-30° C. After completion of the reaction, reaction mass was filtered to obtain(S)-2-((tert-butoxycarbonyl)amino)-3-ethoxy-3-oxopropyl2-(4-cyano-2-methoxybenzylidene)-3-oxobutanoate as a mixture of the E/Z isomers (28.0 grams, Yield=64%, Mass: [M+H]⁺: 461.49 which directly used for next step without further purification.

### 3.5 (S)-2-((tert-butoxycarbonyl) amino-3-ethoxy-3-oxopropyl 4-(4-cyano-2-methoxy phenyl)-2, 8-dimethyl-5-oxo-1, 4, 5, 6-tetrahydro-1, 6-naphthyridine-3-carboxylate

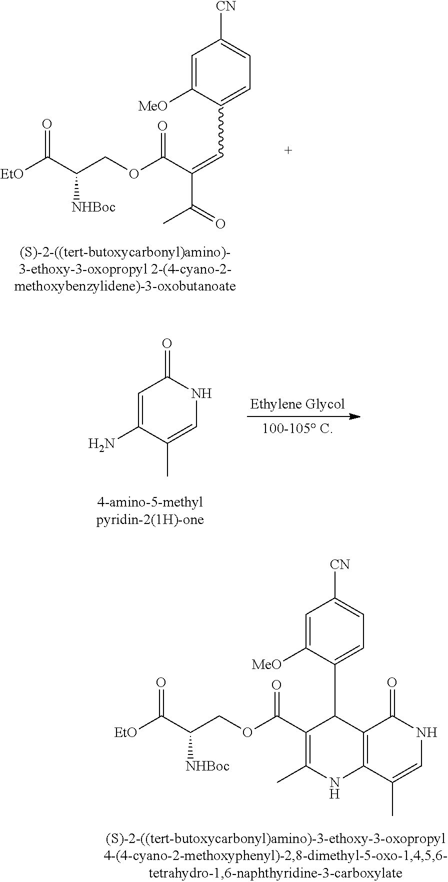

(S)-2-((tert-butoxycarbonyl)amino)-3-ethoxy-3-oxopropyl-2-(4-cyano-2-methoxybenzylidene)-3 oxo butanoate (25.0 grams, 0.054 mmol) and 4-amino-5-methylpyridin-2 (1H)-one (6.73 grams, 0.054 mmol) charged with ethylene glycol (250 ml) in a 500 ml round bottom flask and the reaction mixture heated up to 100-105° C. for 6-8 hours. After reaction completion 500 ml water was charged and stirred for 30 min. Filtered and re-slurried with 500 ml water and stirred for 1 hr. Free flowing reaction mass filtered to provide(S)-2-((tert-butoxycarbonyl) amino-3-ethoxy-3-oxopropyl 4-(4-cyano-2-methoxy phenyl)-2, 8-dimethyl-5-oxo-1,4,5,6-tetrahydro-1,6-naphthyridine-3-carboxylate (20.0 grams, Yield=65%, Mass: [M+H]⁺: 567.62 which directly converted to next step without further purification.

### 3.6 (S)-2-((tert-butoxycarbonyl)amino)-3-ethoxy-3-oxopropyl-4-(4-cyano-2-methoxyphenyl)-5-ethoxy-2, 8-dimethyl-1, 4-dihydro-1, 6-naphthyridine-3-carboxylate

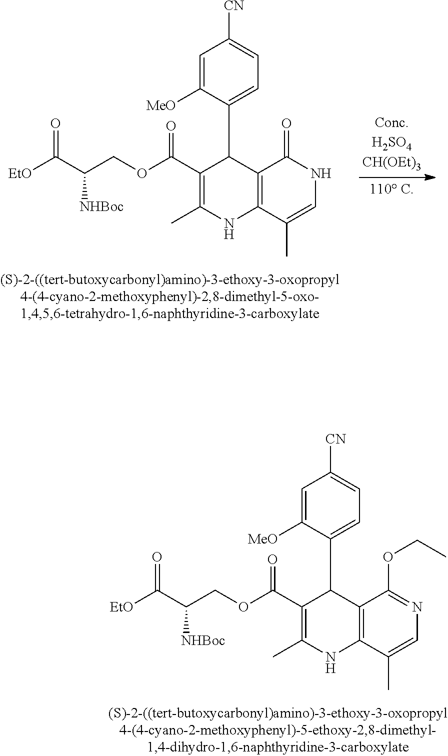 [SMILES: CCOC(=O)[C@H](COC(=O)C1=C(C)Nc2c(C)c[nH]c(=O)c2C1c1ccc(C#N)cc1OC)NC(=O)OC(C)(C)C ; CCOC(=O)[C@H](COC(=O)C1=C(C)Nc2c(C)cnc(OCC)c2C1c1ccc(C#N)cc1OC)NC(=O)OC(C)(C)C]

(S)-2-((tert-butoxycarbonyl)amino)-3-ethoxy-3-oxopropyl-4-(4-cyano-2-methoxyphenyl)-2,8-dimethyl-5-oxo-1,4,5,6-tetrahydro-1,6-naphthyridine-3-carboxylate (15.0 grams, 0.026 mmol) charged in triethyl orthoformate (120 ml) and catalytic amount of concentrated sulphuric acid (0.26 grams, 0.002 mmol) charged; heated to 110-115° C. and stirred for 8 hours. After reaction completion, reaction mass cool down and distilled under vacuum. 200 ml water charged and stirred for 2 hours. After filtration solid was obtained. Obtained solid then extracted with ethyl acetate (200 ml) and layer washed with water (100 ml); collected organic layer dried over anhydrous Na₂SO₄ and filtered. After complete distillation(S)-2-((tert-butoxycarbonyl)amino)-3-ethoxy-3-oxopropyl-4-(4-cyano-2-methoxyphenyl)-5-ethoxy-2,8-dimethyl-1,4-dihydro-1,6-naphthyridine-3-carboxylate obtained as a yellow solid (14.0 grams, Yield=89%, Mass: [M+H]⁺: 595.68 which directly converted to next step without further purification.

### 3.7 (S)-2-((tert-butoxycarbonyl)amino)-3-ethoxy-3-oxopropyl 4-(4-cyano-2-methoxyphenyl)-5-ethoxy-2, 8-dimethyl-1, 6-naphthyridine-3-carboxylate

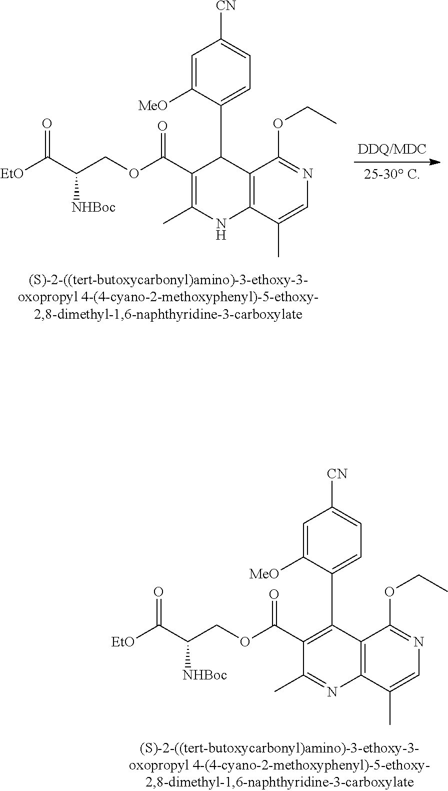 [SMILES: CCOC(=O)[C@H](COC(=O)C1=C(C)Nc2c(C)cnc(OCC)c2C1c1ccc(C#N)cc1OC)NC(=O)OC(C)(C)C ; CCOC(=O)[C@H](COC(=O)c1c(C)nc2c(C)cnc(OCC)c2c1-c1ccc(C#N)cc1OC)NC(=O)OC(C)(C)C]

Small round bottom flask charged with(S)-2-((tert-butoxycarbonyl)amino)-3-ethoxy-3-oxo propyl 4-(4-cyano-2-methoxyphenyl)-5-ethoxy-2,8-dimethyl-1,6-naphthyridine-3-carboxylate (1.30 grams, 0.0021 mmol) in dichloromethane (13 ml). Charged 2,3-Dichloro-5,6-dicyano-1,4-benzoquinone (DDQ) (0.59 grams, 0.0026 mmol) and stirred for 2 hours at 25-30° C. After completion of the reaction; reaction mass filtered and 30 ml water was charged. Layer separated and Organic layer washed with water (4×25 ml) until water become colorless and finally after concentration brownish yellow solid of (S)-2-((tert-butoxycarbonyl)amino)-3-ethoxy-3-oxopropyl 4-(4-cyano-2-methoxyphenyl)-5-ethoxy-2,8-dimethyl-1,6-naphthyridine-3-carboxylate is obtained (0.90 grams, Yield=69.7%, [M+H]⁺: 593.66) which directly converted to next step without further purification.

### 3.8 (S)-2-((tert-butoxycarbonyl)amino)-3-ethoxy-3-oxopropyl(S)-4-(4-cyano-2-methoxy phenyl)-5-ethoxy-2, 8-dimethyl-1,4-dihydro-1,6-naphthyridine-3-carboxylate

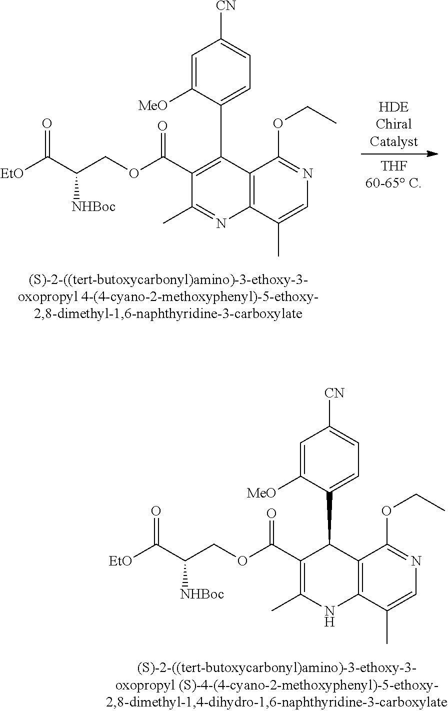 [SMILES: CCOC(=O)[C@H](COC(=O)c1c(C)nc2c(C)cnc(OCC)c2c1-c1ccc(C#N)cc1OC)NC(=O)OC(C)(C)C ; CCOC(=O)[C@H](COC(=O)C1=C(C)Nc2c(C)cnc(OCC)c2[C@@H]1c1ccc(C#N)cc1OC)NC(=O)OC(C)(C)C]

Small round bottom flask charged with(S)-2-((tert-butoxycarbonyl)amino)-3-ethoxy-3-oxopropyl 4-(4-cyano-2-methoxyphenyl)-5-ethoxy-2,8-dimethyl-1,6-naphthyridine-3-carboxylate (250 mg, 0.0004 mmol) in THF (2.5 ml); charged HDE (212 mg, 0.008 mmol) and (S) chiral catalyst (24 mg, 0.00004 mmol) and refluxed for 30 hours at 65-70° C. After completion reaction mass filtered and MLR concentrated under vacuum to obtain oily mass (0.20 grams, Yield=79.7%, [M+H]⁻: 595.68) which directly used for next step without further purification.

### 3.9 S)-2-((tert-butoxycarbonyl)amino)-3-ethoxy-3-oxopropyl(S)-4-(4-cyano-2-methoxy phenyl)-5-ethoxy-2, 8-dimethyl-1,4-dihydro-1,6-naphthyridine-3-carboxylate

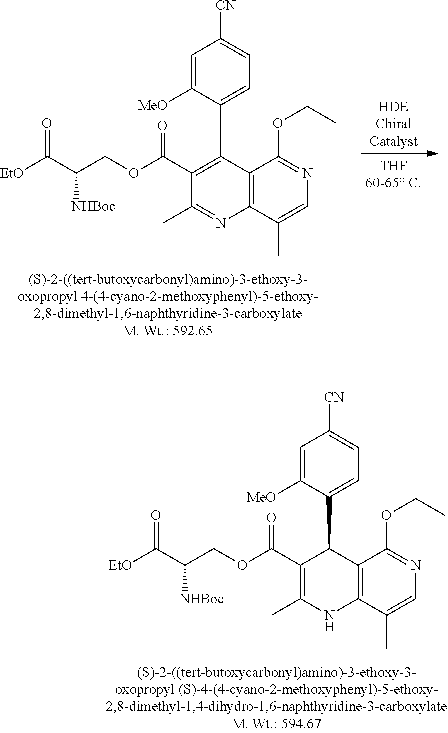 [SMILES: CCOC(=O)[C@H](COC(=O)c1c(C)nc2c(C)cnc(OCC)c2c1-c1ccc(C#N)cc1OC)NC(=O)OC(C)(C)C ; CCOC(=O)[C@H](COC(=O)C1=C(C)Nc2c(C)cnc(OCC)c2[C@@H]1c1ccc(C#N)cc1OC)NC(=O)OC(C)(C)C]

Small round bottom flask charged with(S)-2-((tert-butoxycarbonyl)amino)-3-ethoxy-3-oxopropyl 4-(4-cyano-2-methoxyphenyl)-5-ethoxy-2,8-dimethyl-1,6-naphthyridine-3-carboxylate (250 mg, 0.0004 mmol) in 2-butanol (2.5 ml); charged lot wise HDE (212 mg, 0.008 mmol) and (S) chiral catalyst (24 mg, 0.00004 mmol) and refluxed for 30 hours at 95-100° C. After completion reaction mass filtered and MLR concentrated under vacuum to obtain oily mass.

1. A process for the preparation of a compound of formula III 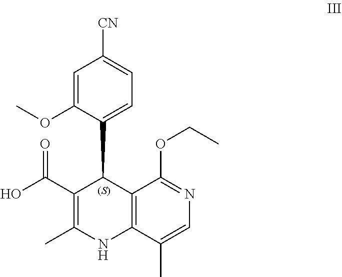
comprising: performing chiral resolution of a compound of formula II 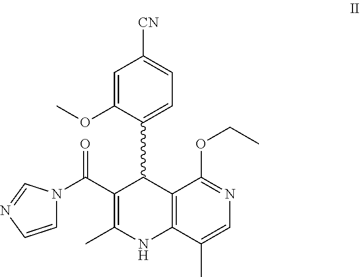 [SMILES: CCOc1ncc(C)c2c1C(c1ccc(C#N)cc1OC)C(C(=O)n1ccnc1)=C(C)N2]
with a chiral acid selected from Di-p-toluoyl-D-tartaric acid or Di-benzoyl-L-tartaric acid.

2. The process according to claim 1, wherein the chiral acid is (+)-Di-p-toluoyl-D-tartaric acid (DTTA) of formula

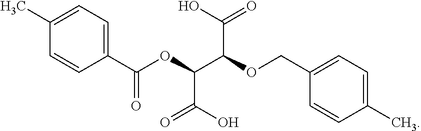 [SMILES: Cc1ccc(CO[C@H](C(=O)O)[C@H](OC(=O)c2ccc(C)cc2)C(=O)O)cc1]

3. The process according to claim 1 comprising reaction steps
(i) reacting a compound of formula II 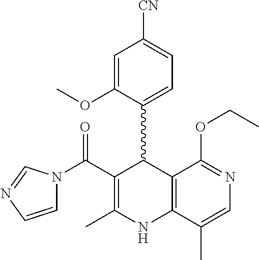 [SMILES: CCOc1ncc(C)c2c1C(c1ccc(C#N)cc1OC)C(C(=O)n1ccnc1)=C(C)N2]
with (+)-Di-p-toluoyl-D-tartaric acid (DTTA) to obtain a compound of formula IIIa 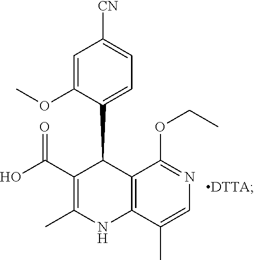
and
(ii) converting the compound of formula IIIa to a compound of formula III 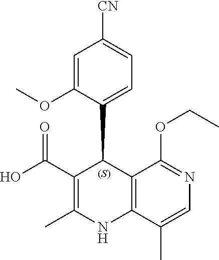
by addition of a base.

4. The process according to claim 3, wherein in step (i) the ratio of the compound of formula II to (+)-Di-p-toluoyl-D-tartaric acid (DTTA) is 1:1 to 1:1.5.

5. The process according to claim 3, wherein in step (i) the reaction is performed in acetone or in a mixture of 2-Methyl-THF and acetone.

6. The process according to claim 5, wherein the ratio of 2-Methyl-THF to acetone is between 1:2 and 1:3.

7. The process according to claim 3, wherein in step (i) the reaction is performed at a temperature between 20-30 degrees Celsius.

8. The process according to claim 3, wherein the compound of formula IIIa obtained in step (i) is isolated by filtration and optionally dried.

9. The process according to claim 3, wherein in step (ii) the base is sodium bicarbonate.

10. The process according to claim 3, wherein in step (ii) the pH is adjusted to a pH of between 6.0-7.5.

11. The process according to claim 3, wherein in step (ii) a mixture of an organic solvent and water is used.

12. The process according to claim 11, wherein in step (ii) a mixture of 2-Methyl THF and water is used.

13. The process according to claim 12, wherein the ratio of 2-Methyl-THF and water is 1:1.

14. The process according to claim 1, wherein the compound of formula III 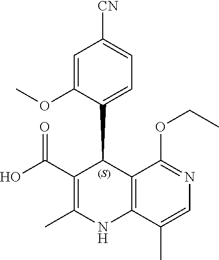
is further converted to Finerenone.
